(19) United States
(12) Patent Application Publication
(10) Pub. No.: US 2010/0168786 A1
(43) Pub. Date: Jul. 1, 2010

(54) SUPPORT FRAME FOR AN EMBOLIC PROTECTION DEVICE

(75) Inventor: Shane Dower, Dungarvan (IE)

Correspondence Address: FULWIDER PATTON, LLP (ABBOTT), 6060 CENTER DRIVE, 10TH FLOOR, LOS ANGELES, CA, 90045, US

(73) Assignee: ABBOTT CARDIOVASCULAR SYSTEMS INC., Santa Clara, CA (US)

(21) Appl. No.: 12/347,672

(22) Filed: Dec. 31, 2008

(51) Int. Cl. A61F 2/06

(52) U.S. Cl. 606/200

(57) ABSTRACT

An embolic filtering device includes a filter support frame which provides a freely articulating structure for deploying and maintaining a filter body in a deployed configuration. A filter body having an inlet end and an outlet end is coupled to the filter support frame. The filter support frame includes a plurality of arms, each arm having a first end and a second end. The first ends of the arms are generally coupled together, with each of the second ends of the arms having a support element coupled thereto. Each arm can articulate independently from another. Each support element can freely articulate on each arm. The free ends of each support element can be biased to an outwardly extending position so that a suitable force will be created to maintain the filter body firmly pressed against a vessel wall when deployed.

### BACKGROUND

The present invention is directed to medical devices generally used during interventional procedures, and more particularly, to a filtering device used to trap and remove foreign particles entrained within the body fluid of a body vessel, such as the vascular system.

In recent years, numerous procedures have been adapted for expanding blood vessels (e.g. arteries), at the positions of lesions in the blood vessels, so that blood can flow through the blood vessels without obstruction from the lesions. In the process of expanding such blood vessels at the positions of the lesions, embolic particles/debris may become detached from the lesions and enter the bloodstream and subsequently migrate through the patient's vasculature to cut off or reduce the amount of oxygenated blood being supplied to sensitive organs such as the brain and heart, which may induce trauma.

Procedures have also been adapted in recent years for preventing embolic debris from flowing through the vessels in the direction of the blood flow. For example, filters have been provided for trapping the emboli. When lesions develop in the carotid artery of a patient, the placement of a filter in the patient's vasculature can somewhat reduce the movement of emboli to blood vessels leading to the patient's brain, thereby preventing strokes.

Such filters are usually delivered in a collapsed position through the patient's vasculature and are then expanded once in place in the patient's blood vessel to trap the emboli. After emboli have been trapped, the filter is collapsed and removed (with the trapped emboli) from the vessel. One particular type of an embolic filter essentially includes a filter body mounted on an associated collapsible support frame which can be collapsed against the guide wire by means of a restraining sheath or catheter for deployment of the filter through a patient's vascular system. Upon retraction of the restraining sheath, the support frame and filter body expand outwardly from the guidewire across a blood vessel within which the filter is positioned to filter blood flowing through the blood vessel.

A practical problem that arises with filter elements of such embolic protection devices is that they should be able to accommodate blood vessels of different diameter as it would be impractical to manufacture a large range of filters each of different size to accommodate all possible diameters of blood vessel. Additionally, not all body vessels have a perfectly concentric diameters. Some have irregular shapes which can be problematic when placing a concentric filter in such an irregular shaped vessel. For example, gaps between the deployed filter and vessel wall could be created which could result in some emboli flowing pass the filter rather than being collected in the filter.

Therefore, in light of the above, it would be desirable for a device and method which can be utilized to treat an occluded vessel and trap any emboli that may be formed during the vascular procedure. Although considerable progress has been made in recent years in providing a satisfactory filter, it would still be desirable to provide a filter which is simple, cost efficient and trustworthy in construction, and can be easily deployed and removed from the patient's vasculature with little or no adverse impact or immunological response to the patient. Such a filtering device should still have a thin profile to reach tight distal lesions in the patient's vasculature and should be capable of being implanted in an irregularly shaped body vessel. Such a filtering device also should be capable of fitting within a number of different sized body vessels.

The present invention is directed towards overcoming these and other problems.

### SUMMARY OF THE INVENTION

The present invention is generally directed to an embolic filtering device for trapping and removing emboli from a body vessel (e.g. an artery) and more specifically directed to a filter support frame which provides a freely articulating structure for deploying and maintaining a filter body in a deployed configuration. In one particular embodiment of the invention, the embolic filtering device includes a filter body having an inlet end and an outlet end. The inlet end of the filter body includes one or more inlet openings sized to allow blood or other bodily fluid and embolic material to enter the filter body. The outlet end of the filter body includes a plurality of outlet openings sized to allow through passage of the blood or other bodily fluids yet small enough to retain undesired embolic material within the filter body. The embolic filtering device further includes a filter support frame coupled to the filter body and movable between a collapsed configuration for movement through a body vessel and an extended outwardly projecting deployed configuration to support the filter body in an expanded configuration. In one aspect, the filter support frame can be biased to the deployed configuration.

The filter support frame includes a plurality of arms, each arm having a first end and a second end. The first ends of the arms are generally coupled together, with each of the second ends of the arms having a support element coupled thereto. Each arm can freely and independently articulate. Additionally, each support element can freely and independently articulate on each arm. The free ends of each support element also can be biased to an outwardly extended position so that a suitable force will be created to maintain the filter body firmly pressed against the vessel wall when deployed.

In one aspect, the support elements of each arm cooperatively define a segmented hoop when placed in the deployed configuration. Each support element is coupled to the filter body to maintain the filter body in direct contact with the wall of the body vessel. Since the support elements on each arm are unattached to any other portion of the filter support in order to allow independent articulation, each arm and support element are capable of independently expanding radially outward to contact the wall of the body vessel to maintain the filter body in a deployed configuration. Accordingly, each arm and support element can articulate, as is needed, in order to fit within an irregularly shaped body vessel. Likewise, due to the independent movement of each arm and associated support element, the filter support frame is capable of easily expanding into various body lumens having different diameters. This eliminates the need to match filters to particular vessel sizes.

In one aspect of the invention, the arms of the filter support frame are biased to the deployed configuration and are held in the collapsed configuration by a restraining sheath. Once the restraining sheath is removed, the arms will expand radially outward. The support elements on the arms will then abut against the wall of the vessel to provide a tight seal which prevents fluid and emboli from passing between the filter body and the vessel wall. When the device is to be collapsed and removed from the patient, the physician simply moves a recovery catheter back over the embolic filtering device to cause the arms to collapse, along with the filter body, thereby trapping any collected emboli in the filter body. The lengths of the arms and the filter body should be sufficiently long so that the embolic filtering device traps the emboli deep within the distal end of the filter body. This helps prevent any backflow of trapped emboli into the vessel when the embolic filtering device is being collapsed.

In other aspects of the present invention, the lengths of the support element can vary from one another. Also, the lengths of the arms can be varied so that the support elements are staggered from each other, but are close enough to each other to maintain the filter body in the deployed configuration. Each arm can include bend points which enhance the ability of the arm to articulate at certain regions along the arm.

In another aspect, the support elements can be configured to wrap around a center element, such as a guide wire or a tubular member, upon which the filter support frame is mounted. In this fashion, each support element will be capable of bending around this mounting element in order to reduce the overall profile of the embolic filtering device.

After the embolic filtering device has been placed at the target location in the body vessel, an interventional device can be disposed in the vessel to treat a lesion or other abnormality in the body vessel. Any suitable interventional device can be used with the present invention. After the interventional device has performed the procedure, the embolic filtering device can be collapsed and removed from the vessel. Emboli created during the interventional procedure are released into the fluid flow (e.g. bloodstream) and are trapped within the filtering assembly.

These and other advantages of the present invention will become more apparent from the following detailed description of the invention, when taken in conjunction with the accompanying exemplary drawings.

### BRIEF DESCRIPTION OF THE DRAWINGS

FIG. 1 is a perspective view showing one particular embodiment of an embolic filtering device embodying features of the present invention;

FIG. 2 is a perspective view showing only the filter support frame of FIG. 1;

FIG. 3 is a side-elevational view of a delivery system which delivers the embolic filtering device shown in FIG. 1 within a body vessel, such as an artery;

FIG. 4 is a side-elevational view of the embolic filtering device shown in FIG. 1 deployed within an artery with a stent delivery catheter placing a stent at a target lesion;

FIG. 5 is a cross-sectional view showing the embolic filtering device of FIG. 1 within an irregularly shaped body vessel;

FIG. 6 is a perspective view showing another embodiment of a filter support made in accordance with the present invention; and

FIG. 7 is a side elevational view showing the filter support of FIG. 6 in a collapsed configuration with the support elements wrapped around a tubular support member.

### DETAILED DESCRIPTION OF THE PREFERRED EMBODIMENTS

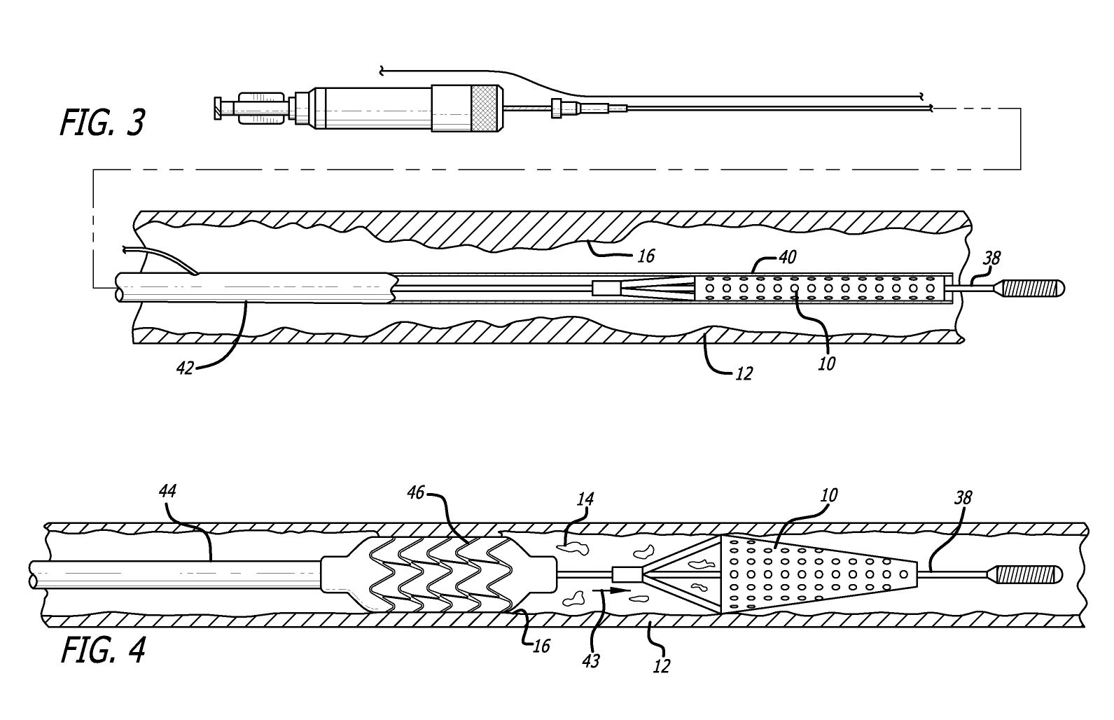

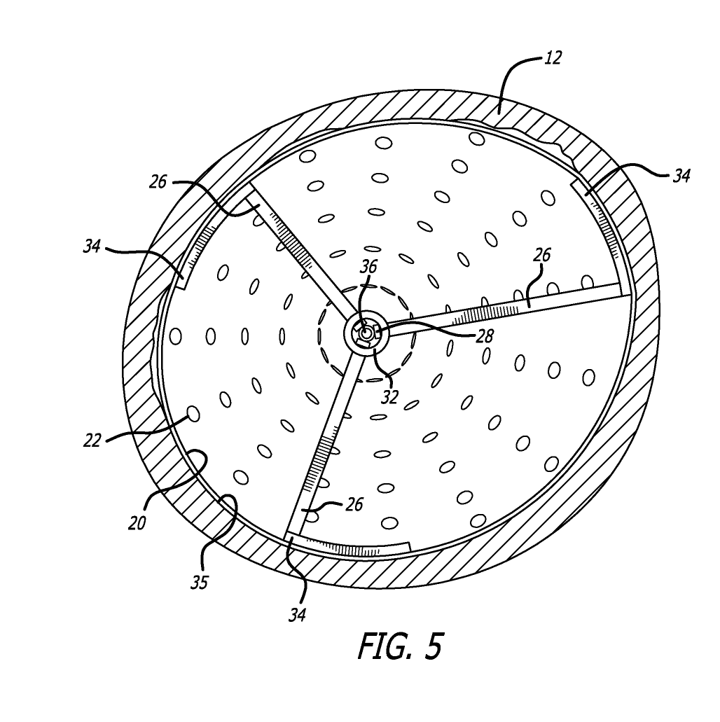

An embolic filtering device generally indicated at 10 and constituting one preferred embodiment of the present invention traps and removes emboli from a body vessel. The embolic filtering device 10 is adapted to be delivered in a body vessel, such as a blood vessel 12, in a collapsed configuration (FIG. 3) to pass the body fluid (for example, blood) in the vessel and block the passage of emboli 14 (FIGS. 4 and 5) in the fluid. The emboli 14 are produced when the blood vessel 12 is treated at the position of a lesion 16 during an interventional procedure such as, a balloon angioplasty procedure, a stenting procedure, an atherectomy procedure and the like. The present invention is designed to collect and remove such embolic debris from the artery to prevent the blockage of the smaller vessels downstream from the area of treatment. When the interventional procedure is being performed in the carotid arteries, the embolic filtering device 10 is especially adapted to prevent blockage of small blood vessels leading to the brain which, if blocked, can result in the patient suffering a stroke.

The embolic protection device 10 includes a filter body 18 made from a mesh or suitable filtering material. The filter body 18 includes an inlet end 20 and an outlet end 22, the inlet end 20 of the filter body 18 having one or more inlet openings sized to allow blood and embolic material to enter the filter body, the outlet end of the filter body having a plurality of outlet openings sized to allow through passage of blood but to retain undesired embolic material within the filter body. For example, the filter body 18 may be made from a thin polymer having small openings to pass the fluid in the vessel 12 while blocking the passage of the emboli 14 in the fluid. The filter member 18 may be coated with an anti-thrombotic agent to minimize blockage of the filter media by thrombosis. The filter material could also be non-porous. The ability of the embolic protection filter to deploy and retract multiple times could allow the physician to periodically retract the device to allow blood flow downstream from the filter. Such non-porous materials would include polymeric materials well-known in medical catheter design.

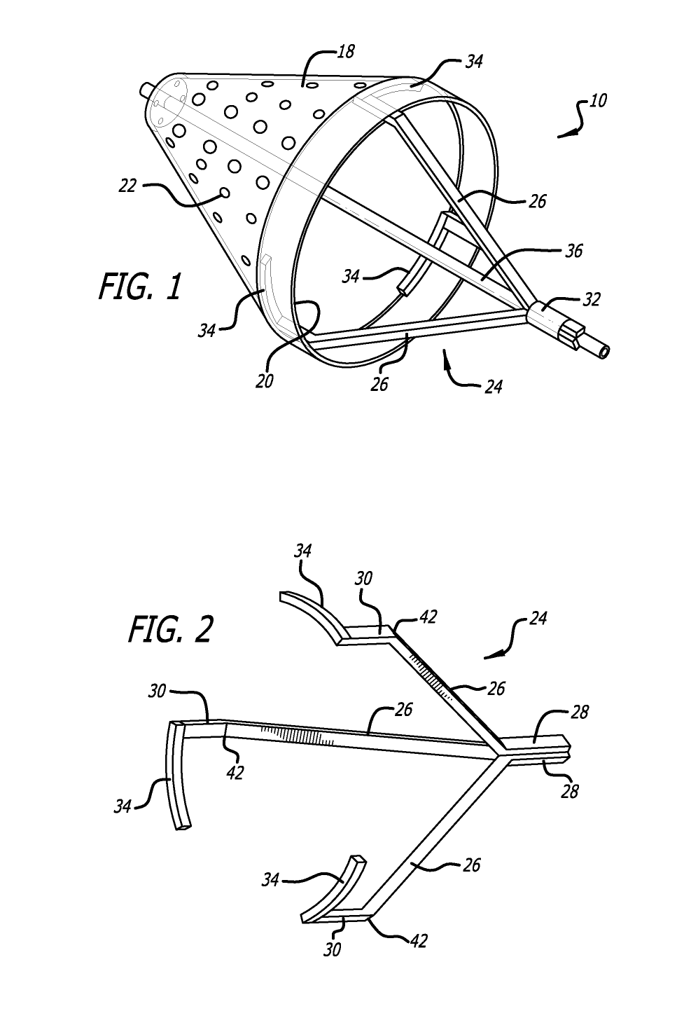

The device 10 further includes a filter support frame 24, which in this particular embodiment, is coupled to the filter body 18. The filter support frame 24 includes a plurality of arms 26 which are movable between a collapsed configuration (FIG. 3) for movement through a body vessel and an extended outwardly projecting deployed configuration (FIGS. 1 and 4) to support the filter body 18 in an expanded configuration. The filter support frame 24 can be biased to the deployed configuration. Each arm 26 has a first end 28 and a second end 30. Each of the first ends 28 of the arms 26 can be coupled together, as shown in FIGS. 1 and 4. A ring or band 32 can be used to couple the first ends 28 together.

Each arm 26 has a support element 34 coupled at the second end 30. In the particular embodiment disclosed in FIGS. 1 and 2, these support elements 34 cooperatively define a segmented hoop when the filter support frame 24 is placed in the deployed configuration. Each support element 34 is coupled to the filter body 18 to achieve apposition of the filter body 18 with the wall 35 of the body vessel, as is shown in FIGS. 4 and 5. These support elements 34 provide sufficient support to maintain the filter body 18 firmly pressed against the vessel wall 35. Additionally, the free end of the support element can be biased radially outward from its associated arm to provide a suitable force on the inside portion of the filter body 18 in order to maintain the filter body firmly placed against the vessel wall, as is shown in FIGS. 4 and 5. In this regard, the free end of the support element can be biased to extend radially from the plane of the arm to create a stronger biasing force which will act against the filter body. Each support element may have a slight radius of curvature set into its length of the element to match the curvature of the body lumen.

In the particular embodiment shown in FIG. 1, the filter body 18 and filter support frame 24 are mounted to a tubular support member 36 which, in turn, can be slidably disposed on a guide wire 38 (FIGS. 3 and 4). This tubular support member 36 may be made from a material such as nickel titanium (NiTi) alloy or any other material which has sufficient axial strength and flexibility to move the filter device 10 along the guide wire into tortuous anatomy. For example, a hypotube made from Nitinol would be suitable since it possess sufficient strength, has adequate flexibility to be advanced through tortuous anatomy, and can be made with a thin wall to reduce the overall profile of the device. This material also is advantageous because it is able to withstand buckling.

Alternatively, the tubular support member 36 could be eliminated with the filter body 18 and filter support frame 24 carried directly by the guide wire 38. The filter body 18 and filter support frame 24 could either be slidingly carried on the guide wire or fixedly attached to the guide wire. One way to fixedly attach the filter support frame 24 to the guide wire is to use stops placed on the guide wire to prevent longitudinal motion of the first ends of the arms of the filter support frame 24. Alternatively, the first ends of the arms could be bonded to the guide wire. The support elements 34 can be formed so as to wrap around this tubular support member 36 or guide wire 38 when placed in the collapsed configuration. The wrapping of these support members results in the embolic filtering device having a lower profile when placed in the collapsed position.

In the collapsed configuration, the embolic protection device 10 could be disposed within a sheath which forms a portion of a delivery catheter 40. In the deployed configuration, the arms 26 would flare radially outward to allow the support elements 36 to engage the wall 35 of the vessel 12. The arms 26 may be formed from a material having shape memory which causes the arms 26 to expand against the wall of the vessel 12 when the filtering assembly 22 is released from the sheath 20. Nitinol is one suitable material which could be used to create the arms 26. Each of the arm 26 may further include one or more bend points 42 which allows the arm to more easily articulate. For example, a bend point 42 could be placed near the second end 30 of the arm, as is shown in FIGS. 1 and 2. Additional bend points 42 could be used along the length of the arm to provide additional articulation. The bend point 42 could simply be a predetermined bend placed on the arm which will allow the arm to generally flex in two dimensions. Additionally, the bend point could be a notch formed into the arm which provides three degrees of articulation at the bend point.

The embolic protection device 10 is initially provided in the collapsed position within the sheath 40 as is shown in FIG. 3. As a first step, the device 10 is disposed in the vessel 12 to a position past the lesion 16 in the direction of the fluid flow (downstream of the lesion 16). When the device 10 has been properly positioned in the vessel 12, the sheath 40 is moved in a direction to expose the filter body 18. This causes the arms 26 and the filter body 18 to expand against the wall 35 of the vessel 12. The expansion of the device 10 into the deployed configuration is shown in FIG. 4. In this configuration, the filter body 18 passes the fluid in the vessel 12 but blocks the passage of the emboli 14 in the fluid. The direction of the fluid flow in the vessel 12 is indicated schematically by solid arrows 43.

An interventional device, for example, a stent delivery catheter 44 and a stent 46 can be utilized to treat the lesion 16 and open up the artery 12 to increase blood flow therethrough. This stent delivery catheter 44 and the stent 46 may be constructed in a manner well known in the art. It should be appreciated that other interventional devices can be used with the embolic protection device. For example, balloon expandable stents, self-expanding stents, balloon angioplasty catheters, atherectomy devices and the like can be used to treat the stenosis as well.

The stent delivery catheter 44 and stent 46 are then positioned in the vessel 12 across the lesion 16. This is shown in FIG. 4. The stent 46 expands against the wall of the vessel 12, thereby opening the vessel at the position of the lesion 16 to increase the flow of fluid through the vessel. The expansion of the stent 46 against the wall of the vessel 12 is indicated in FIGS. 4 and 5. The lesion 16 will thus be compressed against the wall 35 of the vessel 12, thereby expanding the opening in the vessel at the position of the lesion 16.

The expansion of the stent 46 against the lesion 16 may create emboli 14 as is shown in FIG. 4. However, emboli 14 are blocked by the filter body 18 from flowing through the mesh. When all of the emboli 14 have flowed through the filter body 18, the stent delivery catheter 48 can be withdrawn.

As is shown in FIG. 5, the blood vessel 12 may be irregularly shaped in that the vessel 12 is not completely circular. However, since the arms 26 are not attached to each other and are independently articulating, individual arms can extend out radially as needed in order to maintain the filter body 18 in proper contact with the vessel wall 35. If the support elements 34 were connected together, a circular hoop would be created which would not fit perfectly within the irregularly shaped vessel 12 and could cause large gaps to form between the filter body 18 and the vessel wall 35. However, since each arm 26 deploys independently from the other arms, individual arms could expand further against the wall 35 than the other arms to prevent the formation of gaps.

The embolic protection device 10 (with the trapped embolic debris retained by the filter body 18) can then be removed in a direction to dispose the device back into a recovery catheter (not shown).

As can be seen in the embodiment of FIGS. 1 and 2, each support element 34 extends substantially perpendicular to the arm 26 to which it is attached. The individual support elements 34 cooperate to form a segmented hoop when the support frame 24 is placed in the deployed configuration. Since a segmented hoop is created, rather than a solid hoop, the support elements 34 and arms 26 are allowed to freely articulate independently from each other. Again, this independently articulating support frame 24 can more easily configure itself to an irregular shaped vessel and possesses the ability to configure itself to many different sized body vessels.

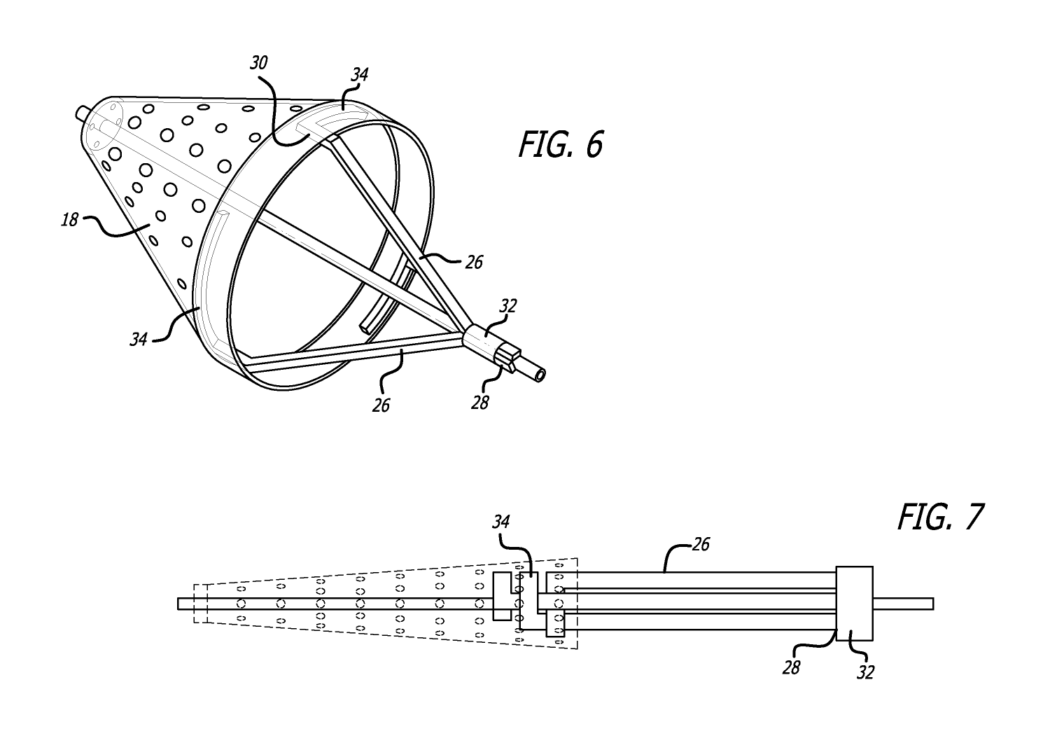

Each of the support elements 34 do not have to have the same length, but rather, can have varying lengths, as is shown in the embodiment of FIGS. 6 and 7. In this particular embodiment, the different lengths of the support elements still cooperate to provide the necessary articulation to maintain the filter body in the fully deployed configuration. Additionally, each of the arms 26 can have different lengths which allow the support elements 34 to be staggered relative to one another. This staggered configuration of support elements will create an offset, segmented hoop as is shown in FIG. 6. The support elements do not lie in the same plane with each other, but are nevertheless remain close enough to created this offset, segmented hoop configuration. Since the support elements 34 are capable of wrapping around the tubular support member or guide wire, this staggered arrangement will prevent the support elements from wrapping around each other in the collapsed configuration. Accordingly, this staggered arrangement can reduce the overall profile of the embolic filtering device when placed in a collapsed configuration.

It should be appreciated that the lengths of the arms and support elements 34 can vary, depending upon the particular application. It should be understood that while three arms 26 are shown forming the support frame 24, more or less arms could be used to form the frame 24. Additionally, the arms 26 are shown generally evenly spaced (120° apart) from each other in the embodiments disclosed herein. However, it is possible to space the arms unevenly. Also, although the support segments are shown disposed substantially perpendicular to its associated arm, it is possible to align the support element at non-perpendicular angles with the axis of the arm.

The arms of the filter support frame of the disclosed embodiments are shown as generally having a rectangular cross section. It should be appreciated that the arms could have different cross sections such as, but not limited to, square or circular cross sections. For example, the arms could be made from circular wire, which would create a circular cross section.

The filter support frame of the present invention can be made in many ways. However, one preferred method of making the filter support frame is to cut a thin-walled tubular member, such as nickel-titanium hypotube, to remove portions of the tubing in the desired pattern for each arm and support element, leaving relatively untouched the portions of the tubing which are to form each arm and support element. It is preferred to cut the tubing in the desired pattern by means of a machine-controlled laser. Alternatively, each arm can be made separately and treated to have a “set” imparted into the structure in order to allow the arm to be biased to the expanded configuration. The support element could be created integrally with each arm or could be made separately and coupled to the arm using welding, adhesives and other well known bonding techniques.

Materials which could be utilized to create the filter support frame include nickel-titanium alloy, such as Nitinol, or other shape-memory materials. It is also possible to utilize stainless steel to form the frame as well. Stainless steel may be alloy-type: 316L SS, Special Chemistry per ASTM F138-92 or ASTM F139-92 grade 2. Special Chemistry of type 316L per ASTM F138-92 or ASTM F139-92 Stainless Steel for Surgical Implants in weight percent. Other suitable metallic and alloy materials could also be used. Polymeric materials also could be used.

The size of the arm and support element is usually very small, so the tubing from which it is made must necessarily also have a small diameter. Typically, the tubing has an outer diameter on the order of about 0.020-0.040 inches in the unexpanded condition. The wall thickness of the tubing is about 0.076 mm (0.003-0.006 inches). For embolic filtering devices implanted in body lumens, such as PTA applications, the dimensions of the tubing maybe correspondingly larger. While it is preferred that the filter support frame be made from laser cut tubing, those skilled in the art will realize that the strut assembly can be laser cut from a flat sheet and then rolled up in a cylindrical configuration with the longitudinal edges welded to form a cylindrical member.

Polymeric materials which can be utilized to create the filter body include, but is not limited to, polyurethane and Gortex, a commercially available material. Other possible suitable materials include ePTFE. Braided metallic and alloyed materials such as nitinol could also be used to form the filter body. The material can be elastic or non-elastic. The wall thickness of the filter body can be about 0.001-0.005 inches. The wall thickness may vary depending on the particular material selected. As can be seen in FIGS. 1 and 6, the filter body includes a proximal, collar-like structure which is attached directly to the support elements. This structure allows the filter body to open within the body vessel without forming gaps between the vessel wall and filter body. For this reason, this proximal collar-like portion of the filter body may have a larger wall thickness than the remainder of the filter body to provide additional stiffness in this region. However, additional stiffness in this region will not prevent the filter body from folding into the collapsed configuration. The material forming the filter body can be made into the desired shape utilizing, for example, blow-mold technology. The perfusion openings can be any different shape or size. A laser, a heated rod or other process can be utilized to create to perfusion openings in the filter material. The holes, would of course be properly sized to catch the particular size of embolic debris of interest. Additionally, the filter material can have a “set” put in it much like the “set” used in dilatation balloons to make the filter element expand to a desired shape and re-wrap more easily when placed in the collapsed position.

Although this invention has been disclosed and illustrated with reference to particular preferred embodiments, the principles involved are susceptible for use in numerous other embodiments which will be apparent to persons of ordinary skill in the art. Various modifications and improvements can be made to the invention without departing from the scope thereof. Additionally, although individual features of one embodiment of the invention may be discussed herein or shown in the drawings of the one embodiment and not in other embodiments, it should be apparent that individual features of one embodiment may be combined with one or more features of another embodiment or features from a plurality of embodiments. The invention is, therefore, to be limited only as indicated by the scope of the appended claims.

I claim:

1. An embolic protection filter, comprising:
a filter body having an inlet end and an outlet end, the inlet end of the filter body having one or more inlet openings sized to allow blood and embolic material to enter the filter body, the outlet end of the filter body having a plurality of outlet openings sized to allow through passage of blood but to retain undesired embolic material within the filter body; and
a filter support frame coupled to the filter body and movable between a collapsed configuration for movement through a body vessel and an extended outwardly projecting deployed configuration to support the filter body in an expanded configuration, the filter support frame being biased to the deployed configuration, the filter support including a plurality of arms, each arm having a first end and a second end, the first end of each arms being coupled together, each second arm having a support element extending therefrom;
wherein when placed in the deployed configuration position, the support element of each arm cooperatively defining a segmented hoop, each support element being coupled to the filter body to achieve apposition of the filter body with a wall of the body vessel.

2. A filter as claimed in claim 1, wherein each support element extends substantially perpendicular to the arm to which is coupled when the filter support frame is placed in the deployed configuration.

3. A filter as claimed in claim 2, wherein the length of at least one support element is longer than another support element.

4. A filter as claimed in claim 2, wherein each support element has a radius of curvature.

5. A filter as claimed in claim 1, wherein in the deployed configuration, each support element extends partially circumferentially.

6. A filter as claimed in claim 1, further including a bend point formed near each second end of each arm.

7. A filter as claimed in claim 1, wherein each support element include a free end which is biased to a radially extended position.

8. A filter as claimed in claim 7, further including a bend point located at the junction of each support element and its associated arm.

9. A filter as claimed in claim 6, wherein a portion of each arm contacts a portion of the filter body.

10. A filter as claimed in claim 9, wherein the portion of the arm which contacts the filter body extends between the second end of each arm and a bend point located on the arm.

11. A filter as claimed in claim 1, wherein each arm articulates independently from the other arms.

12. An embolic protection filter, comprising:
a filter body having an inlet end and an outlet end, the inlet end of the filter body having one or more inlet openings sized to allow blood and embolic material to enter the filter body, the outlet end of the filter body having a plurality of outlet openings sized to allow through passage of blood but to retain undesired embolic material within the filter body; and
a filter support coupled to the filter body and movable between a collapsed configuration for movement through a body vessel and an extended outwardly projecting deployed configuration to support the filter body in an expanded configuration, the filter support being biased to the deployed configuration, the filter support including a plurality of arms, each arm having a first end and a second end defining a length with at least one arm having a different length from the other arms, the first end of each arms being coupled together, each second end having a support element extending therefrom;
in the deployed configuration position, the support element of each arm cooperatively defines an offset, segmented hoop, each support element being coupled to the filter body to achieve apposition of at least part of the filter body with a wall of the body vessel.

13. A filter as claimed in claim 12, further including a bend point formed near each second end of each arm.

14. A filter as claimed in claim 12, wherein in the deployed configuration, each support element extends partially circumferentially.

15. A filter as claimed in claim 12, wherein the length of at least one support element is longer than another support element.

16. A filter as claimed in claim 12, wherein each support element include a free end which is biased to a radially extended position.

17. A filter as claimed in claim 12, further including a bend point formed near each second end of each arm.

18. A filter as claimed in claim 12, further including a bend point located at the junction of each support element and its associated arm.

19. A filter as claimed in claim 1, wherein the filter support frame and filter body are mounted to a tubular support member and the support elements are adapted to wrap around the tubular support member when the filter support frame is placed in the collapsed configuration.

20. A filter as claimed in claim 1, wherein the filter support frame and filter body are carried by a guide wire and the support elements are adapted to wrap around the guide wire when the filter support frame is placed in the collapsed configuration.
